(19) United States
(12) Patent Application Publication
(10) Pub. No.: US 2010/0104703 A1
(43) Pub. Date: Apr. 29, 2010

(54) COOKER AND METHOD FOR CONTROLLING THE SAME

(76) Inventors: Ri Na HWANG, Seoul (KR); Seong Ho Cho, Seoul (KR); Young Min Lee, Seoul (KR)

Correspondence Address: KED & ASSOCIATES, LLP, P.O. Box 221200, Chantilly, VA, 20153-1200, US

(21) Appl. No.: 12/607,224

(22) Filed: Oct. 28, 2009

(30) Foreign Application Priority Data
Oct. 28, 2008 (KR) 10-2008-0105721

(51) Int. Cl. A47J 27/00; G01N 33/02

(52) U.S. Cl. 426/231; 99/325

(57) ABSTRACT

Provided are a cooker and a method for controlling the same. The cooker includes a cooking chamber in which a food is cooked and a heating source providing heat for cooking the food within the cooking chamber. The heating source maintains a temperature of the cooking chamber at a preset first set temperature, and then maintains a preset second set temperature at a temperature exceeding the first set temperature.

### CROSS-REFERENCE TO RELATED APPLICATIONS

The present application claims priority under 35 U.S.C. 119 and 35 U.S.C. 365 to Korean Patent Application No. 10-2008-0105721 (filed on Oct. 28, 2009), which is hereby incorporated by reference in its entirety.

### BACKGROUND

The present disclosure relates to a cooker, and more particularly, to a cooker and a method for controlling the same.

Cookers are cooking equipment that cooks foods using electricity or gas. Such a cooker includes a cooking chamber in which foods are cooked and a heater used as a heating source for cooking the foods within the cooking chamber. When a meat is cooked using the cooker, an outer surface of the meat is heated to brown the outer surface thereof while the meat is heated at a high temperature to prevent gravies contained in the meat from leaking. Then, when the outer surface of the meat is brown, the meat is heated at a low temperature to boil the inside of the meat.

There are, however, the following limitations when the meat is cooked using a related art.

In the related art, a meat is heated at a high temperature to brown an outer surface of the meat in order to prevent gravies contained in the meat from leaking during a meat cooking process. Thus, harmful components such as fats, etc., do not leak from the meat to the outside.

Also, when the outer surface of the meat is heated at the high temperature to brown the outer surface thereof, and then, the inside of the brown meat is heated at the low temperature, the outer surface of the meat may be burned, and the inside of the meat may be undercooked. Thus, the meat may not be completely cooked.

### SUMMARY

Embodiments provide a cooker in which harmful components contained in a meat are discharged to the outside during a cooking process and a method for controlling the same.

Embodiments also provide a cooker in which a meat is well cooked and a method for controlling the same.

In one embodiment, a cooker comprises: a cooking chamber in which a food is cooked; and a heating source providing heat for cooking the food within the cooking chamber, wherein the heating source maintains a temperature of the cooking chamber at a preset first set temperature, and then maintains a preset second set temperature at a temperature exceeding the first set temperature.

In other embodiment, a method for controlling a cooker, the method comprises: operating a heating source to maintain a temperature of a cooking chamber at a preset first set temperature, thereby cooking a food; and operating the heating source to maintain the temperature of the cooking chamber at a second set temperature preset as a temperature value exceeding the first set temperature.

In another embodiment, a method for controlling a cooker including a cooking chamber in which a food is cooked and a heating source providing heat for cooking the food within the cooking chamber, the method comprising: transmitting an input signal for setting at least one of an operation beginning of the heating source, a kind of food for setting a target temperature of the food, and a cooked degree of the food to an input part; operating the heating source to maintain a temperature of the cooking chamber at a preset first set temperature until the temperature of the cooking chamber reaches a temperature obtained by reduction at a rate of less than about 90% from the target temperature; and when a temperature of the food reaches a preset reference temperature, operating the heating source to maintain the temperature of the cooking chamber at a second set temperature preset as a temperature value exceeding the first set temperature until the temperature of the temperature of the food reaches the target temperature.

The details of one or more embodiments are set forth in the accompanying drawings and the description below. Other features will be apparent from the description and drawings, and from the claims.

### BRIEF DESCRIPTION OF THE DRAWINGS

FIG. 1 is a schematic block diagram of a cooker which is applied in a process of controlling the cooker according to a first embodiment.

FIG. 2 is a flowchart illustrating a process of controlling the cooker according to the first embodiment.

FIG. 3 is a graph illustrating a reduction rate of a fat in case where a meat is cooked using the process of controlling the cooker according to the first embodiment and in case where the meat is cooked using a related art.

FIG. 4 is a graph illustrating a reduction amount of a fat in case where a meat is cooked using the process of controlling the cooker according to the first embodiment and in case where the meat is cooked using a related art.

FIG. 5 is a graph illustrating the difference of a nucleic acid amount in case where a meat is cooked using the process of controlling the cooker according to the first embodiment and in case where the meat is cooked using a related art.

FIG. 6 is a flowchart illustrating a process of controlling a cooker according to a second embodiment.

FIG. 7 is a flowchart illustrating a process of controlling a cooker according to a third embodiment.

FIG. 8 is a flowchart illustrating a process of controlling a cooker according to a fourth embodiment.

### DETAILED DESCRIPTION OF THE EMBODIMENTS

Hereinafter, configurations of a cooker which is applied in a method for controlling the cooker according to a first embodiment will be described in detail with reference to the accompanying drawings.

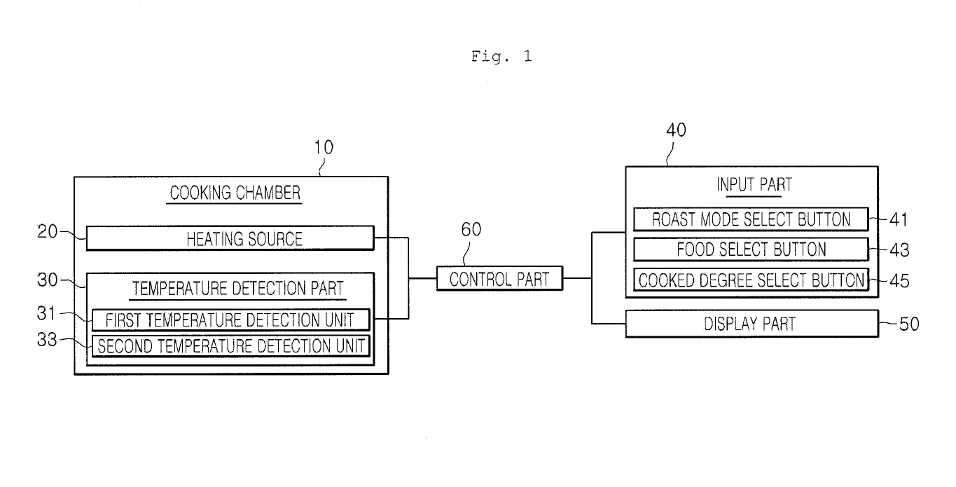

FIG. 1 is a schematic block diagram of a cooker which is applied in a process of controlling the cooker according to a first embodiment.

Referring to FIG. 1, a cooker according to this embodiment includes a cooking chamber 10, a heating source 20, a temperature detection part 30, an input part 40, a display part 50, and a control part 60.

The cooking chamber 10 provides a space in which foods are cooked. The heating source 20 provides heat for cooking the foods within the cooking chamber 10. For example, the heating source 20 may include a heater, a convection device, and a magnetron.

The temperature detection part 30 includes a first temperature detection unit 31 and a second temperature detection unit 33. The first temperature detection unit 31 detects an internal temperature of the cooking chamber 10, and the second temperature detection unit 33 detects an internal temperature of the foods that are cooked within the cooking chamber 10.

The input part 40 receives an input signal for operating the cooker. Furthermore, the input part 40 includes a roast mode select button 41, a food select button 42, and a cooked degree select button 45. The roast mode select button 41 receives an input signal for selecting a roast mode. Here, the roast mode represents a mode in which the meat is cooked according to the first embodiment. The food select button 43 receives an input signal for selecting a kind of meat to be cooked within the cooking chamber 10 in a state where the roast mode select button 43 receives the input signal for selecting the roast mode. Also, the cooked degree select button 45 receives an input signal for selecting an cooked degree (for example, one of well-done, medium, and rare) of the meat to be cooked within the cooking chamber 10 in a state where the food select button 43 receives the input signal for selecting the kind of meat. For another example, the cooked degree select button 45 may receive an input signal for adjusting an amount of water to be used for cooking.

The display part 50 displays various information with respect to operations of the cooker. For example, the display part 50 may display whether the roast mode is selected, a selected kind of meat, and a selected cooked degree.

The control part 60 controls the heating source 20 according to the input signal transmitted into the input part 40 and a food temperature detected by the first and second temperature detection parts 31 and 33 and controls the display part 50 to display these related information. In detail, the control part 60 calculates first and second set temperature within the cooking chamber 10 and a target temperature of the food according to the input signals transmitted into the food select button 43 and the cooked degree select button 45 and controls the heating source 20 to cook the foods within the cooking chamber 10 as described below.

Hereinafter, a method for controlling a cooker according to a first embodiment will be described in detail with reference to the accompanying drawings.

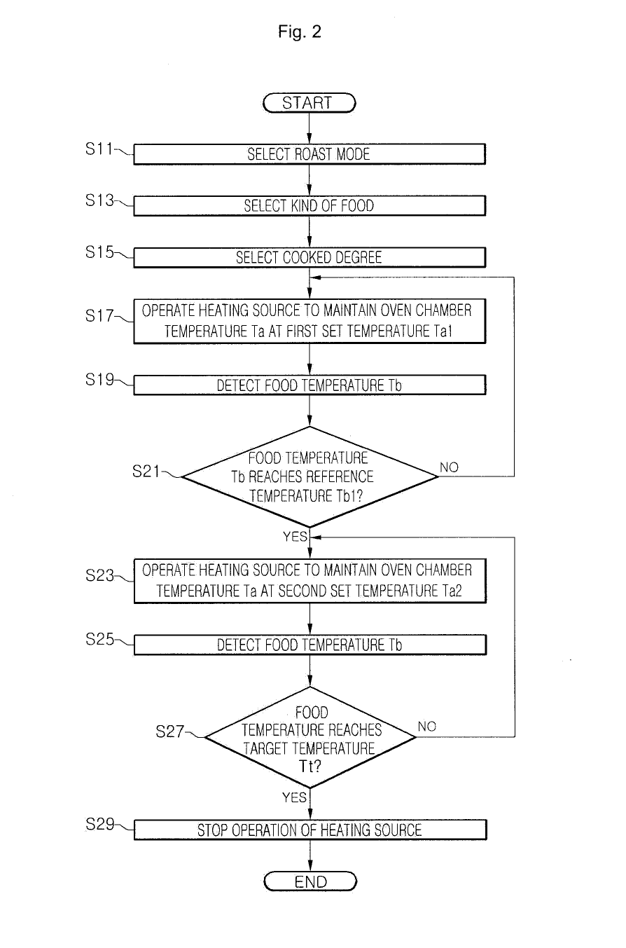

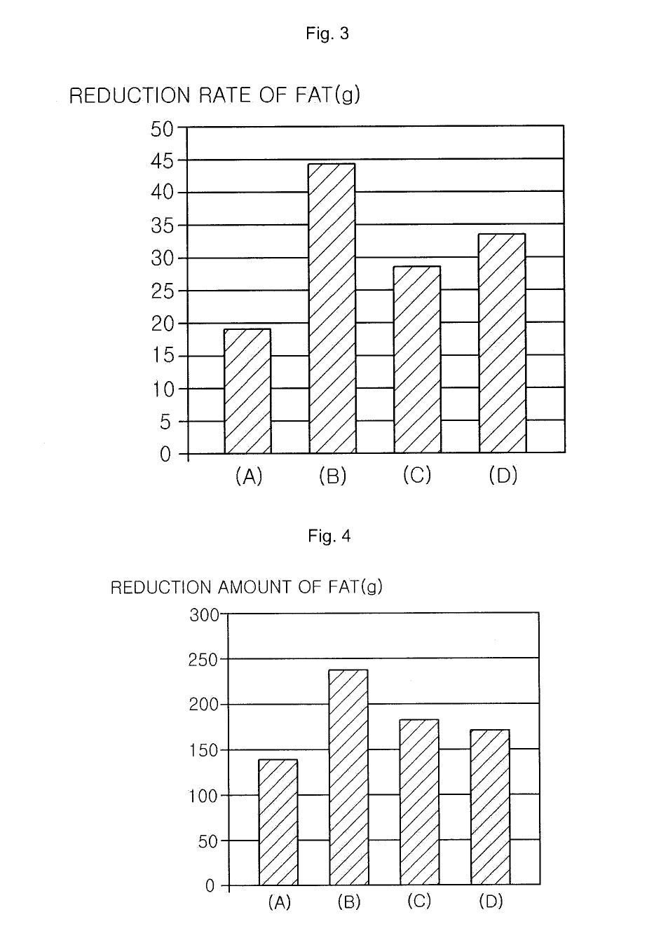

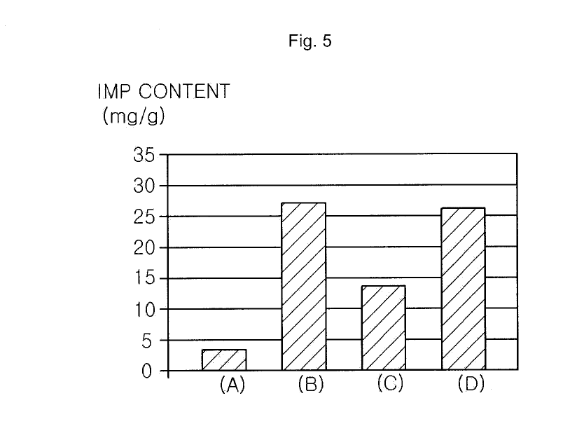

FIG. 2 is a flowchart illustrating a process of controlling the cooker according to the first embodiment, and FIG. 3 is a graph illustrating a reduction rate of a fat in case where a meat is cooked using the process of controlling the cooker according to the first embodiment and in case where the meat is cooked using a related art. FIG. 4 is a graph illustrating a reduction amount of a fat in case where a meat is cooked using the process of controlling the cooker according to the first embodiment and in case where the meat is cooked using a related art, and FIG. 5 is a graph illustrating the difference of a nucleic acid amount in case where a meat is cooked using the process of controlling the cooker according to the first embodiment and in case where the meat is cooked using a related art.

Referring to FIG. 2, a roast mode select button 41 receives an input signal for selecting a roast mode in operation S11. A food select button 43 receives an input signal for selecting a kind of food, i.e., meat in operation S13 in a state where the roast mode select button 41 receives the input signal for selecting the roast mode in operation S11. When the food select button 43 receives the input signal for selecting the kind of food in operation S13, a cooked degree select button 45 receives an input signal for selecting a cooked degree of the meat in operation S15.

In operation S17, a heating source 20 is operated to cook the food, i.e., the meat within a cooking chamber 10. The heating source 20 supplies heat into the cooking chamber 10 to maintain a cooking chamber temperature Ta at a preset first set temperature Ta1. The inside of the food within the cooking chamber 10 is cooked in a state where the heating source 20 is operated to maintain the cooking chamber temperature Ta at the first set temperature Ta1. At this time, a first temperature detection unit 31 detects the cooking chamber temperature Ta.

When the heat source 20 is operated to cook the food within the cooking chamber 10 in operation S17, a temperature detection part 30 detects a food temperature Tb in operation S19. For example, a probe used as the temperature detection part 30 may detect the food temperature Tb.

In operation S19, a control part determines whether the food temperature Tb detected by the temperature detection part 30 reaches a reference temperature Tb1. Here, the reference temperature Tb1 is determined according to a final target temperature Tt of the food preset by the kind of meat and the cooked degree selected depending on the input signals transmitted into the food select button 43 and the cooked degree select button 45 in operations S13 and S15. At this time, the reference temperature Tb1 is lower than the target temperature Tt. For example, the reference temperature Tb1 may be determined as a temperature obtained by reduction at a preset rate from the target temperature Tt.

When the food temperature Tb reaches the reference temperature Tb1 in operation S21, the heating source 20 is operated to maintain the cooking chamber temperature Ta at a preset second set temperature Ta2 in operation S23. Here, the second set temperature Ta2 may be set to a relatively high temperature than the first set temperature Ta1. As described above, due to the heating operation in operation S21, the inside of the food within the cooking chamber 10 is heated, and also, the outer surface of the food is heated to brown the outer surface thereof.

When the heating source 20 is operated in operation 23, the temperature detection part 30 detects the food temperature Tb in operation S25. In operation S27, the control part 60 determines whether the food temperature Tb detected by the temperature detection part 30 in operation S23 reaches the target temperature Tt.

When the food temperature Tb reaches the target temperature Tt in operation S27, the operation of the heating source 20 is stopped in operation S29. Thus, the cooking operation of the cooker is completed.

An applicant of the present disclosure performed tests for cooking a food under various conditions using the method for controlling the cooker according to an embodiment. In detail, a ribeye of about 1.8 kg (about 3 pound) was cooked with the target temperature Tt of about 60° C. Also, the ribeye was cooked according to following four cases.

(A) a case in which a set temperature is about 160° C.

(B) a case in which the first and second set temperatures Ta1 and Ta2 are about 80° C. and about 220° C., respectively, and the reference temperature Tb1 is about 80% (about 48° C.) of the target temperature Tt.

(C) a case in which the first and second set temperatures Ta1 and Ta2 are about 80° C. and about 220° C., respectively, and the reference temperature Tb1 is about 90% (about 54° C.) of the target temperature Tt.

(D) a case in which the first and second set temperatures Ta1 and Ta2 are about 100° C. and about 220° C., respectively, and the reference temperature Tb1 is about 80% (about 48° C.) of the target temperature Tt.

Referring to FIGS. 3 and 4, it is seen that a rate and amount of a fat reduced during the cooking process increase in the cases (B) to (D) when compared to the case (A). That is, the fat of about 19.5% and about 138.3 g decreases in the case (A), but the fat of about 44.2% and about 234.7 g decreases in the case (B). Also, the fat of about 28.7% and 181.6 g and the fat of about 33.3% and about 168.7 g decrease in the case (C) and (D), respectively.

Referring to FIG. 5, a nucleic acid (e.g., inosinic 5′-monophosphate (IMP)) that is one of components by which the taste of meat is decided in the cooking process is relatively well preserved in the cases (B) to (D) when compared to the case (A). Thus, it is seen that a large amount of nucleic acid is contained in the meat. That is, the meat contains the IMP of about 4.23 mg/g in the case (A), but contains the IMP of about 27.33 mg/g in the case (B). Also, the meat contains the IMP of about 14.60 mg/g and the IMP of about 27.13 mg/g in the cases (C) and (D), respectively.

Hereinafter, a method for controlling a cooker according to a second embodiment will be described in detail with reference to the accompanying drawings.

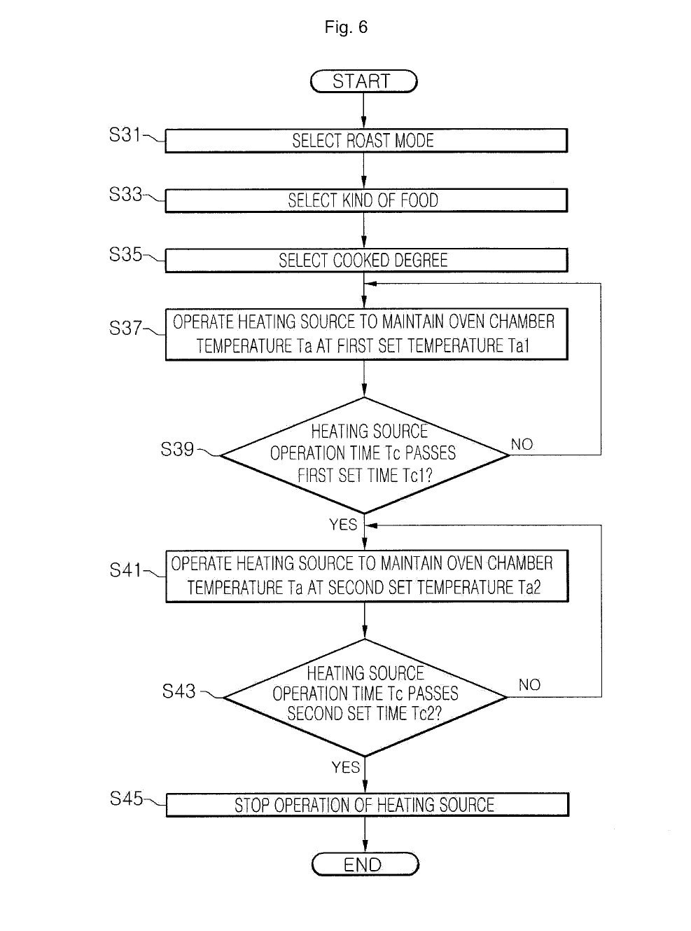

FIG. 6 is a flowchart illustrating a process of controlling a cooker according to a second embodiment.

Referring to FIG. 6, since operations S31 to S37 are equal to the operations S11 to S17 of the above-described first embodiment, their detailed descriptions will be omitted.

When a heating source 20 is operated in operation S37, a control part 60 controls whether a heating source operation time Tc passes a first set time Tc1 in operation S39. The first set time Tc1 is set to a time for which a food within a cooking chamber 10, i.e., the inside of a meat is cooked by the cooking source 20 operated to maintain a cooking chamber temperature Ta at the first set temperature Ta1 in operation S37. For example, the first set time Tc1 may range from about 150 minutes to about 210 minutes. Particularly, the first set time Tc1 may be set to a time ranging from about 160 minutes to about 180 minutes. Alternately, the first set time Tc1 may be set to a time for which the heating source 20 is operated until a food temperature Tb reaches a reference temperature Tb1. Here, the reference temperature Tb1 is set to a temperature equal to that of the above-described first embodiment.

When the control part 60 determines that the heating source operation time Tc passes the first set time Tc1 in operation S39, the control part 60 operates the heating source 20 to maintain the cooking chamber temperature Ta at a second set temperature Ta2 in operation S41. At this time, the second set temperature Ta2 is set to a temperature equal to that of the above-described first embodiment.

In operation S43, the control part 60 determines whether the heating source operation time Tc passes the second set time Tc2. Here, the second set time Tc2 is set to a time for which a food within a cooking chamber 10, i.e., the inside of a meat is cooked by the cooking source 20 operated to maintain a cooking chamber temperature Ta at the second set temperature Ta2 in operation S37. For example, the second set time Tc2 may range from about 20 minutes to about 60 minutes that is a relatively short time when compared to the first set time Tc1. For another example, the second set time Tc2 may be set to a time for which the heating source 20 is operated until a food temperature Tb reaches a target temperature Tt. Here, the target temperature Tt is set to a temperature equal to that of the above-described first embodiment.

When the control part 60 determines that the heating source operation time Tc passes the second set time Tc2 in operation S43, the control part 60 stops the operation of the heating source 20 in operation S45. Thus, the cooking operation of the cooker is completed.

Hereinafter, a method for controlling a cooker according to a third embodiment will be described in detail with reference to the accompanying drawings.

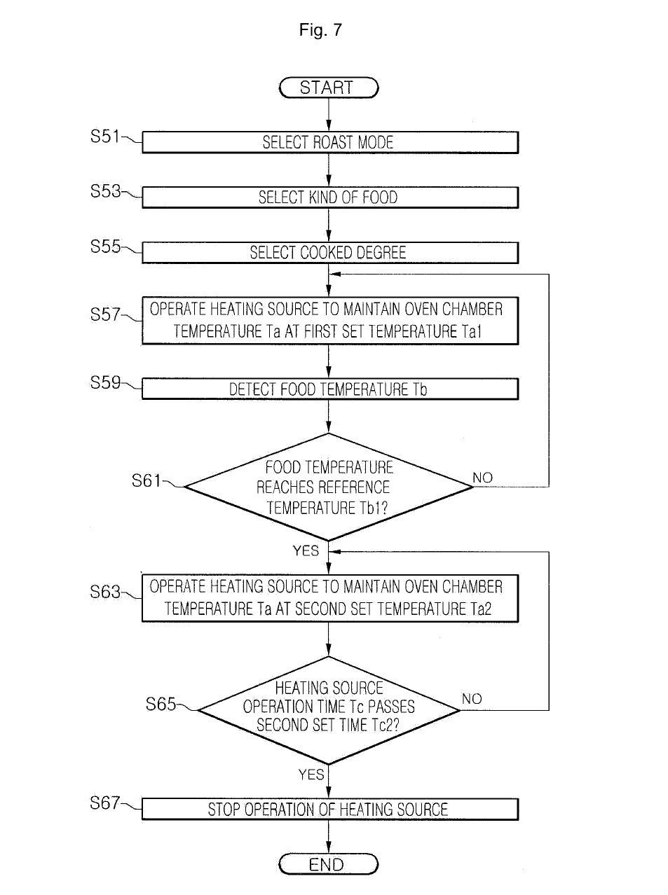

FIG. 7 is a flowchart illustrating a process of controlling a cooker according to a third embodiment.

Referring to FIG. 7, since operations S51 to S57 are equal to the operations S11 to S19 of the above-described first embodiment, their detailed descriptions will be omitted.

In operation S61, a control part 60 determines whether a food temperature Tb detected by a temperature detection part 30 reaches a reference temperature Tb1. Here, the reference temperature Tb1 may be set to a temperature equal to that of the above-described first embodiment.

When the control part 60 determines that the food temperature Tb reaches the reference temperature Tb1 in operation S61, the control part 60 operates a heating source 20 in operation S63. Here, the control 60 operates the heating source 20 to maintain a cooking chamber temperature Ta at a second set temperature Ta2.

In operation S65, the control part 60 determines whether a heating source operation time Tc passes a set time Tc2. Here, the set time Tc2 is set to a time equal to that of the second embodiment.

When the heating source operation time Tc passes the set time Tc passes the set time Tc2 in operation S5, the control part 60 stops the operation of the heating source 20 in operation S67. Thus, the cooking operation of the cooker is completed.

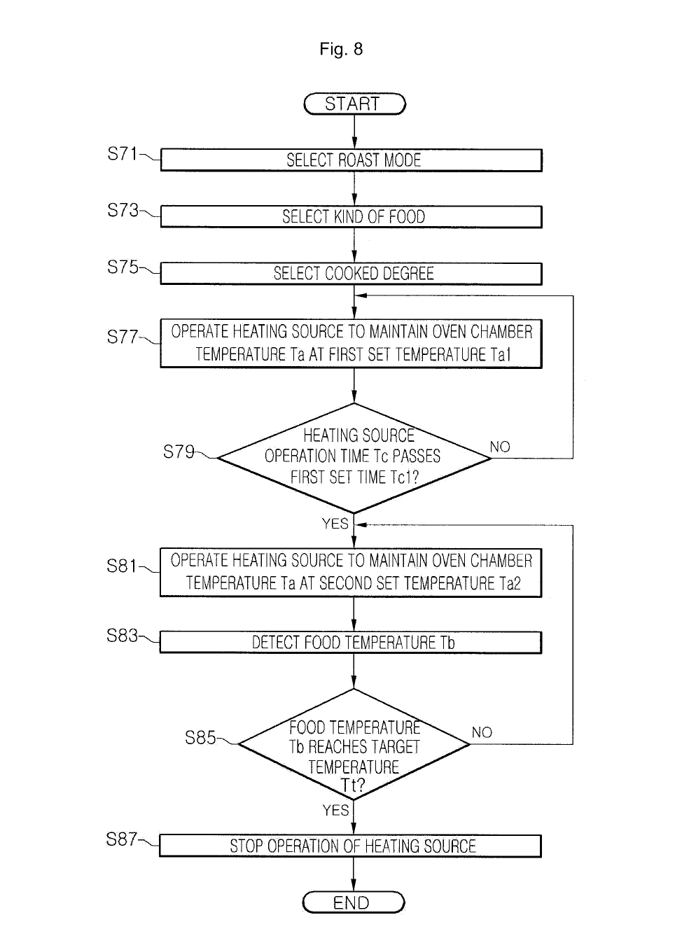

FIG. 8 is a flowchart illustrating a process of controlling a cooker according to a fourth embodiment.

Referring to FIG. 8, since operations S71 to S77 are equal to the operations S11 to S17 of the above-described first embodiment, their detailed descriptions will be omitted.

When a heating source 20 is operated in operation S77, a control part 60 determines whether a heating source operation time Tc passes a set time Tc1 in operation 79. Here, since the set time Tc1 may be set to a time equal to that of the second embodiment, their detailed descriptions will be omitted.

When the heating source operation time passes the set time Tc1 in operation S79, the control part 60 operates the heating source 20 to maintain a cooking chamber temperature Ta at a second set temperature Ta2 in operation S81. When the heating source 20 is operated in operation S79, a temperature detection part 30 detects a food temperature within a cooking chamber 10 in operation S83.

In operation S85, the control part 60 determines whether the food temperature Tb detected in operation S83 reaches a target temperature Tt. Here, the target temperature Tt may be set to a temperature equal to that of the first embodiment.

When the food temperature Tb reaches the target temperature Tt in operation S85, the control part 60 stops the operation of the heating source 20 in operation S87. Thus, the cooking operation of the cooker is completed.

As described above, in case where the food is cooked according to the second to fourth embodiments, it may expect that a significant reduction rate of the fat, a significant reduction amount of the fat, and a significant nucleic acid content when compared to a relate art equal to that of the above-described first embodiment.

According to the embodiments, the harmful components contained in the meat may be discharged, and the meat may be well cooked.

As described above, in the cooker according to the embodiments and the method for controlling the same, the following effects may be expected.

The meat is heated at the low temperature, and then, the meat is heated at the high temperature. Thus, the harmful components such as the fat within the meat may be discharged.

Also, the meat is heated at the low temperature to cook the meat as a whole, and then, the meat is heated at the high temperature to brown the outer surface thereof. Thus, since the meat is not locally cooked or burned, the meat may be well cooked.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A cooker comprising:
a cooking chamber in which a food is cooked; and
a heating source providing heat for cooking the food within the cooking chamber,
wherein the heating source maintains a temperature of the cooking chamber at a preset first set temperature, and then maintains a preset second set temperature at a temperature exceeding the first set temperature.

2. The cooker according to claim 1, wherein the heating source is operated to maintain the temperature of the cooking chamber at the first set temperature, and then, when a temperature of the food reaches a preset reference temperature, the heating source is operated to maintain the temperature of the cooking chamber at the second set temperature.

3. The cooker according to claim 2, wherein the reference temperature is set to a temperature value of less then about 90% of a target temperature of the food preset according to a kind of food.

4. The cooker according to claim 1, wherein the heating source is operated to maintain the temperature of the cooking chamber at the first set temperature, and then, when a temperature of the food reaches a preset reference temperature, the heating source is operated to maintain the temperature of the cooking chamber at the second set temperature, and
the heating source is operated to maintain the temperature of the cooking chamber at the second set temperature, and then, when the temperature of the food reaches a preset target temperature, an operation of the heating source is stopped.

5. The cooker according to claim 4, wherein the reference temperature is set to a temperature less than the target temperature.

6. The cooker according to claim 1, wherein the heating source is operated for a first set time to maintain the temperature of the cooking chamber at the first set temperature, and then, the heating source is operated for a second set time to maintain the temperature of the cooking chamber at the second set temperature.

7. The cooker according to claim 6, wherein the first set time is set to a time for which the heating source is operated until the temperature of the food reaches a temperature equal to that of about 90% of a target temperature of the food preset according to a kind of food, and
the second set time is set to a time for which the heating source is operated until the temperature of the food reaches the target temperature.

8. The cooker according to claim 1, wherein the first set temperature is set to a temperature of less than about 100° C., and
the second set temperature is set to a temperature of greater than about 200° C.

9. The cooker according to claim 1, wherein the second set temperature is set to a temperature exceeding at least about 80° C. when compared to the first set temperature.

10. The cooker according to claim 1, further comprising an input part receiving an input signal for setting at least one of an operation beginning of the heating source, a kind of food, and a cooked degree of the food.

11. A method for controlling a cooker, the method comprising:
operating a heating source to maintain a temperature of a cooking chamber at a preset first set temperature, thereby cooking a food; and
operating the heating source to maintain the temperature of the cooking chamber at a second set temperature preset as a temperature value exceeding the first set temperature.

12. The method according to claim 11, wherein the heating source is operated to maintain the temperature of the cooking chamber at the first set temperature, and then, when a temperature of the food reaches a preset reference temperature, the heating source is operated to maintain the temperature of the cooking chamber at the second set temperature.

13. The method according to claim 12, wherein the reference temperature is set to a temperature value of less then about 90% of a target temperature of the food preset according to a kind of food.

14. The method according to claim 11, wherein the heating source is operated to maintain the temperature of the cooking chamber at the first set temperature, and then, when a temperature of the food reaches a preset reference temperature, the heating source is operated to maintain the temperature of the cooking chamber at the second set temperature, and
the heating source is operated to maintain the temperature of the cooking chamber at the second set temperature, and then, when the temperature of the food reaches a preset target temperature, an operation of the heating source is stopped.

15. The method according to claim 14, wherein the reference temperature is set to a temperature less than the target temperature.

16. The method according to claim 11, wherein the heating source is operated for a first set time to maintain the temperature of the cooking chamber at the first set temperature, and then, the heating source is operated for a second set time to maintain the temperature of the cooking chamber at the second set temperature.

17. The method according to claim 16, wherein the first set time is set to a time for which the heating source is operated until the temperature of the food reaches a temperature equal to that of about 90% of a target temperature of the food preset according to a kind of food, and
the second set time is set to a time for which the heating source is operated until the temperature of the food reaches the target temperature.

18. The method according to claim 11, wherein the first set temperature is set to a temperature of less than about 100° C., and
the second set temperature is set to a temperature of greater than about 200° C.

19. A method for controlling a cooker including a cooking chamber in which a food is cooked and a heating source providing heat for cooking the food within the cooking chamber, the method comprising:
transmitting an input signal for setting at least one of an operation beginning of the heating source, a kind of food for setting a target temperature of the food, and a cooked degree of the food to an input part;
operating the heating source to maintain a temperature of the cooking chamber at a preset first set temperature until the temperature of the cooking chamber reaches a temperature obtained by reduction at a rate of less than about 90% from the target temperature; and
when a temperature of the food reaches a preset reference temperature, operating the heating source to maintain the temperature of the cooking chamber at a second set temperature preset as a temperature value exceeding the first set temperature until the temperature of the temperature of the food reaches the target temperature.
